(19) United States
(12) Patent Application Publication
(10) Pub. No.: US 2018/0050131 A1
(43) Pub. Date: Feb. 22, 2018

(54) IMPLANTABLE MEDICINE DELIVERY SYSTEMS

(71) Applicant: Washington State University, Pullman, WA (US)

(72) Inventors: Susmita Bose, Pullman, WA (US); Amit Bandyopadhyay, Pullman, WA (US); Dishary Banerjee, Pullman, WA (US); Naboneeta Sarkar, Pullman, WA (US)

(21) Appl. No.: 15/663,221

(22) Filed: Jul. 28, 2017

Related U.S. Application Data
(60) Provisional application No. 62/377,313, filed on Aug. 19, 2016.

(51) Int. Cl. A61L 27/54; A61L 27/58; A61L 27/12; A61L 27/56; A61L 27/34; A61L 27/18; A61L 27/44; B33Y 10/00; B33Y 80/00; A61M 31/00

(52) U.S. Cl. CPC A61L 27/54; A61L 27/58; A61L 27/12; A61L 27/56; A61L 27/34; A61L 27/18; B28B 1/001; B33Y 10/00; B33Y 80/00; A61M 31/002; A61L 2300/416; A61L 2300/216; A61L 2430/02; A61L 27/44

(57) ABSTRACT

Implantable medicine delivery systems, devices, and associated methods are disclosed herein. In one embodiment, a method of enhancing bone regeneration in a human or animal body includes implanting a three-dimensional scaffold in the human or animal body. The three-dimensional scaffold is constructed from a porous ceramic material, a ceramic-polymer composite of a biodegradable ceramic material and a polymer, or a ceramic-polymer-metal composite of a biodegradable ceramic material, a polymer, and a metal and embedded with curcumin in the porous ceramic material, the curcumin at least partially coating an exterior surface of the porous ceramic material. The method also includes directly and controllably releasing the embedded curcumin into a circulatory system of the human or animal body according to a release profile, thereby achieving enhanced bone regeneration in the human or animal body.

### CROSS-REFERENCE TO RELATED APPLICATION(S)

This application is a Non-Provisional Application of and claims priority to U.S. Provisional Application No. 62/377,313, filed on Aug. 19, 2016.

### STATEMENT REGARDING FEDERALLY SPONSORED RESEARCH

This work was supported in part by a grant from the National Institute of Health under Grant/Contract No RO1 AR066361. The government has certain rights in this work.

### BACKGROUND

Plants, fungi, animal parts, and other natural products have been used for medical treatments through much of human history. Modern medicine makes use of many compounds derived from natural products as basis for pharmaceutical drugs. For example, curcumin is a compound derived from rhizomes of turmeric plants. Curcumin has been deemed useful for regulating expression of genes involved in metastasis, cell proliferation, angiogenesis, and osteoclastogenesis. In another example, Aloe Vera has been used for treating various skin conditions.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Applying compounds derived from natural products for medicinal use, however, can face certain difficulties. In one aspect, certain natural compounds can be difficult to absorb by a human or animal body. For example, curcumin has a relatively poor bioavailability in a human or animal body due to low solubility and its hydrophobic nature. In another aspect, maintaining effective concentrations of a natural compound may be difficult due to rapid intestinal and/or liver metabolism. For example, the liver of a human or animal body can quickly metabolize curcumin such that a concentration of curcumin in the blood stream typically decrease precipitately after initial application. The foregoing difficulties can thus restrain applications of curcumin or other natural compounds in practical medicinal use.

Several embodiments of the disclosed technology are directed to manufacture and application of implantable delivery vehicles or devices for controlled release of natural compounds when implanted in a human or animal body. In certain embodiments, a delivery vehicle can include 3-D printed implantable scaffold having a porous ceramic material that carries one or more natural compounds. For instance, one example delivery vehicle can include a scaffold having an interconnected macro porous ceramic material fabricated using hydroxyapatite (“HA”) and/or β-tricalcium phosphate (“β-TCP”) that is coated, impregnated, or embedded with a suitable natural compound. Examples of such natural compound can include curcumin, Aloe Vera, Vitamin D, Vitamin C, or other suitable compounds or compositions derived from natural products.

In other embodiments, the delivery vehicle can also incorporate a polymer matrix containing one or more polymers for influencing bioavailability and/or release profile of the natural compound carried by the ceramic material. For example, in one embodiment, the delivery vehicle can incorporate a polymer matrix as a carrier of the natural compound before being applied to the porous ceramic material. Examples of the polymer matrix can contain one or more of having poly(ε-caprolactone) (“PCL”), poly(lactic-co-glycolic acid) (“PLGA”), and/or poly-ethylene glycol (“PEG”), or other suitable polymeric materials. In further embodiments, the scaffold can also include a barrier layer on top of the applied natural compound carried in the polymer matrix. The barrier layer can include one or more of PCL, PLGA, PEG, or other suitable polymeric materials selected to modify release kinetics of the natural compound carried by the porous ceramic material.

As discussed in more detail later, experiments have shown that certain embodiments of the foregoing delivery vehicle can be applied to controllably release natural compounds in a human or animal body. Such experiments include an in vivo osteogenic bone regeneration study using embodiments of the foregoing delivery vehicle containing curcumin. The experiments revealed enhancement of neo bone formation and accelerated bone healing around a scaffold of curcumin coated porous ceramic material. Further histomorphometric analysis has also substantiated the test data. Thus, the experiments showed that the application of the foregoing example delivery vehicle is suitable to deliver natural compounds in a sustained fashion directly into the circulatory system with beneficial effects, e.g., accelerated bone regeneration and healing.

Several embodiments of the disclosed technology are also directed to adjusting one or more process parameters when manufacturing embodiments of the delivery vehicle to achieve a target release profile of natural compounds when the delivery vehicle is implanted in a human or animal body. In one embodiment, a porosity profile of the porous ceramic material can be adjusted based on a desired release profile of natural compounds. For example, an interior portion of the porous ceramic material may be more (or less) porous than an exterior portion such that the interior portion can carry more (or less) natural compounds. As such, when implanted in a human or animal body, the porous ceramic material can release more (or less) carried natural compounds as time elapses to achieve the desired release profile.

In another embodiment, a polymer chemistry of the polymer matrix that carries the natural compounds can be adjusted to achieve the desired release profile. For example, as discussed in more detail later, PCL and PLGA can affect a release profile of curcumin in different ways when implanted in a human or animal body. In particular, PCL appeared to inhibit burst release of curcumin whereas PLGA appeared to lead to burst release in both an acetate buffer and a phosphate buffer. Thus, by selecting one or more of PCL and PLGA and/or varying a ratio therebetween, a desired release profile from the delivery vehicle can be achieved.

In a further embodiment, one or more of the foregoing and/or other suitable polymers may be used as a barrier layer on the porous ceramic material containing the natural compounds. The natural compounds may be contained in the polymer matrix or may be present without the polymer matrix. Various parameters of the barrier layer can thus be adjusted to achieve a desired release profile. For example, a thickness gradient, chemistry, or spatial distribution of the barrier layer can be adjusted according to the desired release profile. As such, by adjusting one or more of the foregoing process parameters, one can design and fabricate an implantable delivery vehicle having a desired release profile for sustained release of natural compounds.

### BRIEF DESCRIPTION OF THE DRAWINGS

FIG. 1 is a schematic diagram of a manufacturing system for producing a scaffold of a porous ceramic material in accordance with embodiments of the disclosed technology.

FIG. 2 is a block diagram showing computing system software components suitable for the additive deposition system of FIG. 1 in accordance with embodiments of the disclosed technology.

FIGS. 3A-3C are schematic diagrams showing example delivery vehicles in accordance with embodiments of the disclosed technology.

FIGS. 4A-4D are flowcharts showing methods for manufacturing an implantable delivery vehicle for sustained release of natural compounds in accordance with embodiments of the disclosed technology.

FIG. 5 is an example X-Ray Powder Diffraction (“XRD”) pattern of an example HA powder in accordance with embodiments of the disclosed technology.

FIG. 6 is a Fourier transform infrared spectroscopy (“FTIR”) spectra of an example HA powder in accordance with embodiments of the disclosed technology.

FIGS. 7A and 7B are example curcumin release profiles of a) bare, b) PCL-coated, and (c) PLGA-coated HA discs at pH 7.4 for 16 days and 24 hours, respectively, in accordance with embodiments of the disclosed technology.

FIGS. 8A and 8B are example curcumin release profiles of a) bare, b) PCL-coated, and (c) PLGA-coated HA discs at pH 5.0 for 16 days and 24 hours, respectively, in accordance with embodiments of the disclosed technology.

FIGS. 9A-9F illustrate example surface morphology of pure and curcumin coated porous ceramic material at various resolutions in accordance with embodiments of the disclosed technology.

FIGS. 10A-10D illustrate example osteoid like new bone formation and mineralization of newly formed bone formation at an implantable delivery vehicle and host bone interface in accordance with embodiments of the disclosed technology.

FIG. 11 illustrates example histomorphometric analysis results showing enhanced bone formation in curcumin coated porous ceramic material in accordance with embodiments of the disclosed technology.

### DETAILED DESCRIPTION

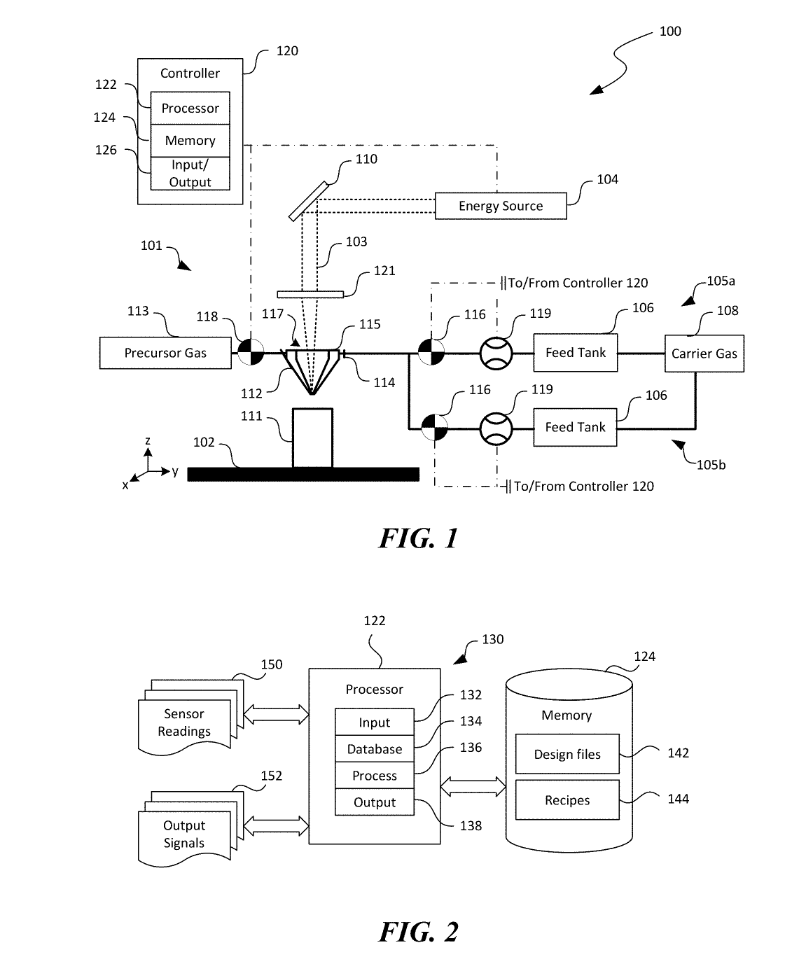

Certain embodiments of systems, devices, articles of manufacture, and processes for delivering natural compounds using implantable delivery vehicles or devices are described below. In the following description, specific details of components are included to provide a thorough understanding of certain embodiments of the disclosed technology. A person skilled in the relevant art will also understand that the disclosed technology may have additional embodiments or may be practiced without several of the details of the embodiments described below with reference to FIGS. 1-11.

As used herein, the term “additive deposition” or “3-D printing” generally refers to a deposition process in which one or more precursor materials are melted by an energy source before being deposited onto a substrate in a line-by-line, layer-by-layer, or section-by-section manner to form a composite product. The formed composite product can have a compositional and/or structural gradient along at least one dimension. For example, a formed composite product can include hydroxyapatite (HA) and β-tricalcium phosphate (β-TCP) with interconnected macro pores. Also used herein, the term “natural compound” generally refers to a chemical compound or a chemical composition derived primarily from a plant, a fungus, an animal part, or other suitable non-artificial sources. Examples of natural compound include curcumin, Aloe Vera, Vitamin D, Vitamin C, Vitamin B-12, and other suitable Vitamins.

FIG. 1 is a schematic diagram of a manufacturing system 100 for producing a scaffold of a porous ceramic material in accordance with embodiments of the disclosed technology. As shown in FIG. 1, the manufacturing system 100 can include a deposition platform 102, an energy source 104, a first feed line 105a, a second feed line 105b, and a controller 120 operatively coupled to one another. Even though particular components are illustrated in FIG. 1, in other embodiments, the manufacturing system 100 can also include power supplies, purge gas supplies, and/or other suitable components.

As shown in FIG. 1, the deposition platform 102 can be configured to carry a substrate having a substrate material (e.g., stainless steel, Ti, etc.) or a formed target product 111 (shown as a cylinder for illustration purposes). The deposition platform 102 can also be configured to move in x-, y-, and z-axis in a raster scan, continuous scan, or other suitable manners. In certain embodiments, the deposition platform 102 can be coupled to one or more electric motors controlled by a logic processor (not shown) to perform various scanning operations. In other embodiments, the deposition platform 102 can be coupled to pneumatic actuators and/or other suitable types of drives configured to perform the scanning operations.

The energy source 104 can be configured to provide an energy stream 103 into a deposition environment 101. In certain embodiments, the energy source 104 can include an Nd:YAG or any other suitable types of laser capable of delivering sufficient energy to the deposition environment 101. In other embodiments, the energy source 104 can also include microwave, plasma, electron beam, induction heating, resistance heating, or other suitable types of energy sources. In the illustrated embodiment, the manufacturing system 100 also includes a reflector 110 (e.g., a mirror) and a focusing lens 121 configured to cooperatively direct the energy stream 103 into the deposition environment 101. In other embodiments, the manufacturing system 100 can also include collimators, filters, and/or other suitable optical and/or mechanical components (not shown) configured to direct and deliver the energy stream 103 into the deposition environment 101. In further embodiments, one or more of the reflector 110 or the focusing lens 121 may be omitted.

The first and second feed lines 105a and 105b can be configured to deliver first and second precursor materials (e.g., metallic powders, ceramic powders, or a mixture thereof) to the deposition environment 101, respectively. In the illustrated embodiment, each feed line 105a and 105b can include a feed tank 106, a valve 116, and a feed rate sensor 119. The valves 116 can each include a gate value, a globe valve, or other suitable types of valves. The feed rate sensor 119 can each include a mass meter, a volume meter, or other suitable types of meter.

The feed tanks 106 can individually include a storage enclosure suitable for storing a corresponding precursor material. The precursor materials can include can include elemental metals (e.g., titanium, aluminum, nickel, silver, etc.) or metal alloys (e.g., stainless steel) to form intermetallic alloys (e.g., VC, Ti/Al₂O₃, TiAl, TiNi, TiAlNi, etc.). In other embodiments, the precursor materials can include ceramic materials (e.g., HA, TCP, BrN2) that can react or otherwise combine with an elemental metal (e.g., Ti) to form high melting point composite materials (e.g., TiBr, TiBr2, TiN, etc.).

In the illustrated embodiment, both the first and second feed lines 105a and 105b are coupled to a carrier gas source 108 containing argon (Ar) or other suitable inert gases. The carrier gas source 108 can be configured to provide sufficient pressure to force the first and second precursor materials from the feed tanks 106 into the deposition environment 101. In other embodiments, each of the first and second feed lines 105a and 105b can include corresponding carrier gas sources (not shown). Even though two feed lines 105a and 105b are shown in FIG. 1 for illustration, in further embodiments, the manufacturing system 100 can include one, three, four, six, eight, or any suitable number of feed lines (not shown).

As shown in FIG. 1, the manufacturing system 100 can also include an optional precursor gas source 113. The precursor gas source 113 can be configured to contain a precursor gas (e.g., nitrogen, oxygen, carbon dioxide, etc.) and provide the precursor gas to the deposition environment 101 via a valve 118. In certain embodiments, the manufacturing system 100 can include more than one precursor gas source 113 containing different precursor gases. In other embodiments, the precursor gas source 113 may be omitted.

In the illustrated embodiment, the manufacturing system 100 includes a deposition head 112 configured to facilitate aligning the precursor materials from the first and/or second feed lines 105a and 105b with the energy stream 103. The deposition head 112 can include one or more feed ports 114 configured to receive the precursor materials from the first and/or second feed lines 105a and 105b or the optional precursor gas from the precursor gas source 113. The deposition head 114 can also include an opening 117 to receive the energy stream 103. In the illustrated embodiment, the deposition head 112 has a generally conical shape such that precursor materials can be exposed to the energy stream 103 at or near a focal point or plane of the energy stream 103. In other embodiments, the deposition head 112 can have other suitable shapes and/or structures. In further embodiments, the deposition head 112 may be omitted. Instead, the first and second precursor materials may be deposited directly onto the deposition platform 102 at or near a focal point or plane of the energy stream 103.

The controller 120 can include a processor 122 coupled to a memory 124 and an input/output component 126. The processor 122 can include a microprocessor, a field-programmable gate array, and/or other suitable logic devices. The memory 124 can include volatile and/or nonvolatile computer readable media (e.g., ROM; RAM, magnetic disk storage media; optical storage media; flash memory devices, EEPROM, and/or other suitable non-transitory storage media) configured to store data received from, as well as instructions for, the processor 122. In one embodiment, both the data and instructions are stored in one computer readable medium. In other embodiments, the data may be stored in one medium (e.g., RAM), and the instructions may be stored in a different medium (e.g., EEPROM). The input/output component 126 can include a display, a touch screen, a keyboard, a track ball, a gauge or dial, and/or other suitable types of input/output devices.

In certain embodiments, the controller 120 can include a computer operatively coupled to the other components of the manufacturing system 100 via a hardwire communication link (e.g., a USB link, an Ethernet link, an RS232 link, etc.). In other embodiments, the controller 120 can include a logic processor operatively coupled to the other components of the manufacturing system 100 via a wireless connection (e.g., a WIFI link, a Bluetooth link, etc.). In further embodiments, the controller 120 can include an application specific integrated circuit, a system-on-chip circuit, a programmable logic controller, and/or other suitable computing frameworks.

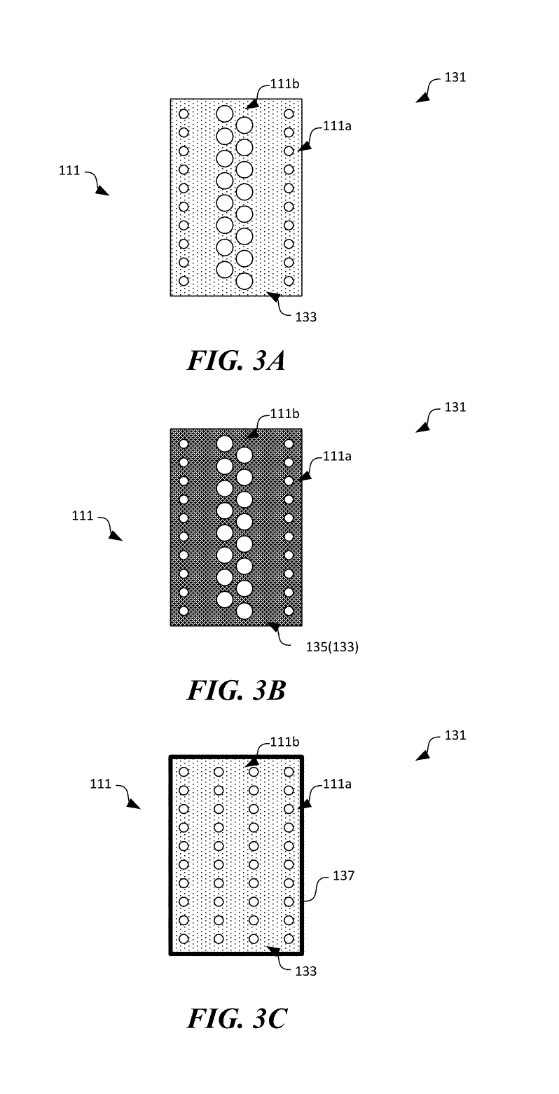

In operation, the controller 120 can receive a desired design file 142 (shown in FIG. 2) for a target product 111 or article of manufacture, for example, an implantable bone replacement. The design file 142 can be in the form of a computer aided design (“CAD”) or other suitable types of file. The design file (or a separate file) 142 can also specify at least one of a composition, a composition gradient, a crystalline structure, a porosity profile, or other desired physical properties for one or more segments of the target product 111. In response, the controller 120 can analyze the design file and generate a recipe having a sequence of operations to form the product via additive deposition in layer-by-layer, section-by-section, or other suitable accumulative fashion.

In one embodiment, the controller 120 can instruct the first and second feed lines 105a and 105b to provide first and/or second precursor materials at spatially and/or temporally varying feed ratios determined based on the design file 142 to the deposition head 112. For example, the feed ratios can be varied along one or more of the x-, y-, or z-axis such that one end of the target product 111 has a first composition (e.g., 100% metal) while the other end of the target product 111 has a second composition (e.g., 100% ceramics). Such composition gradient can be linear, parabolic, elliptical, step-wise, or in other suitable relationship with respect to the x-, y-, or z-axis.

In other embodiments, the controller 120 can also instruct the energy source 104 to provide the energy stream 103 at certain intensity levels to the deposition head 112 to melt the first and second precursor materials, and thus causing the first and second precursor materials to form a composite material having the desired thickness, composition, crystalline structure, or physical properties as specified in the design file. In further embodiments, the energy stream 103 can be at other intensity levels to cause the first and second precursor materials to react by partially melting or without melting the first and/or second precursor materials.

During scanning, the controller 120 can instruct the deposition platform 102 to move the composite material away from the focal point or plane of the energy stream 103 such that the composite material solidifies forming a layer or a portion of the target product 111. In other embodiments, the provided energy stream 103 can also melt a portion of the substrate material (e.g., Ti) of the substrate, thereby causing the substrate material to react with the first and/or second precursor materials to form the composite material. The foregoing operations can then be repeated on the formed layer or portion in, for example, a layer-by-layer manner until the entire product is completed.

In certain embodiments, foregoing deposition operations can be performed in the deposition environment 101 having an inert gas (e.g., argon). The controller 120 can also instruct the valve 118 to open and thus introduce a precursor gas (e.g., nitrogen, oxygen, carbon dioxide, etc.) into the deposition environment 101 when building certain layer or section of the product. The precursor gas can thus at least partially displace the inert gas and react with the first and/or second precursor materials to form a new phase in the product. For example, introducing nitrogen into the deposition environment 101 having a titanium substrate material can form titanium nitride. In another example, introducing carbon dioxide into the deposition environment 101 can form titanium carbide. In other embodiments, the controller 120 can also instruct the energy source 104 to adjust at least one of a laser power or scanning speed based on a desired property for a segment of the product. In further embodiments, the controller 120 can instruct all of the foregoing components of the manufacturing system 100 in any suitable manners.

Unlike CVD, PVD, or thermal spraying techniques, several embodiments of the manufacturing system 100 can be more flexible in achieving a desired transition of compositions, properties, porosity, or other characteristics for the target product 111. For instance, several embodiments of the manufacturing system 100 can be flexible in structural, compositional, dimensional, and property control during deposition by dynamically varying, for example, feed rates or feed ratio of the first and/or second precursor materials, by introducing the precursor gas, by adjusting at least one of power or scanning speed of the energy source 104, and/or manipulating other suitable operating parameters.

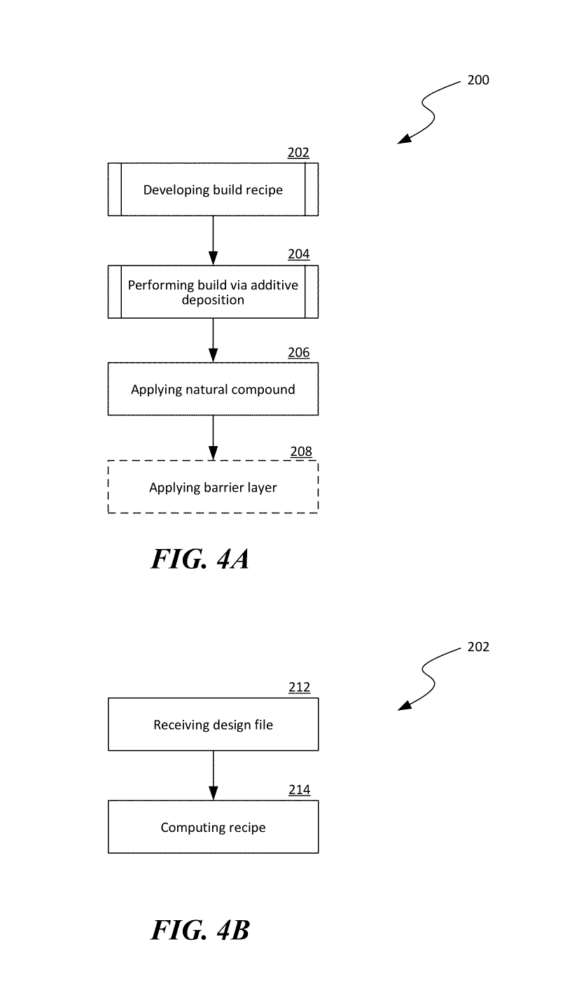

The formed target product 111 can include a three-dimensional scaffold that is loaded with a natural compound by, for example, introducing a desired amount of a solution containing the natural product into the surface and/or interior of the target product 111 to form a delivery vehicle 131 (shown in FIGS. 3A-3C). The delivery vehicle 131 is implantable in a human or animal body. In certain embodiments, the solution may include a polymer matrix containing the natural product. Example polymer matrices may contain PCL, PLGA, PEG, or other suitable polymeric materials. In other embodiments, a barrier layer 137 (shown in FIG. 3C) containing PCL, PLGA, PEG, or other suitable polymeric materials may optionally be deposited onto the target product 111 after being loaded with the natural product to form a delivery vehicle 131. As discussed in more detail herein, one or more of process parameters may be adjusted in order to achieve a target release profile for the natural product carried by the delivery vehicle 131.

FIG. 2 is a block diagram showing computing system software components 130 suitable for the controller 120 in FIG. 1 in accordance with embodiments of the present technology. Each component may be a computer program, procedure, or process written as source code in a conventional programming language, such as the C++ programming language, or other computer code, and may be presented for execution by the processor 122 of the controller 120. The various implementations of the source code and object byte codes may be stored in the memory 124. The software components 130 of the controller 120 may include an input component 132, a database component 134, a process component 136, and an output component 138.

In operation, the input component 132 may accept an operator input, such as a design file for the product in FIG. 1, and communicates the accepted information or selections to other components for further processing. The database component 134 organizes records, including design files 142 and recipes 144 (e.g., steering and/or lane variability), and facilitates storing and retrieving of these records to and from the memory 124. Any type of database organization may be utilized, including a flat file system, hierarchical database, relational database, or distributed database, such as provided by a database vendor such as the Oracle Corporation, Redwood Shores, Calif. The process component 136 analyzes sensor readings 150 from sensors (e.g., from the feed rate sensors 119) and/or other data sources, and the output component 138 generates output signals 152 based on the analyzed sensor readings 150.

FIGS. 3A-3C are schematic diagrams showing example delivery vehicles 131 in accordance with embodiments of the disclosed technology. As shown in FIG. 3A, the delivery vehicle 131 can include an implantable scaffold such as the target product 111 of FIG. 1 constructed from a porous ceramic material, a ceramic-polymer composite of a biodegradable ceramic material and a polymer, or a ceramic-polymer-metal composite of a biodegradable ceramic material, a polymer, and a metal having interconnect macro pores (shown as circles). The biodegradable ceramic material can include hydroxyapatite (“HA”), β-tricalcium phosphate (“β-TCP”), calcium silicate, calcium sulfate, or other suitable ceramic materials. The polymer can include poly(β-caprolactone), poly(lactic-co-glycolic acid), poly-ethylene glycol, or other suitable biodegradable polymeric materials. The metal can include magnesium, iron, zinc, or other suitable bio-resorbable metals or alloys thereof. In the particular example shown in FIG. 3A, the target product 111 can include a generally cylindrical shape with an exterior portion 111a having pores of a first size and an interior portion 111b having pores of a second size larger than the first size. In other examples, the first and second sizes can be generally the same, or can have other suitable relationships. In further examples, the target product 111 can have other suitable shapes, sizes, or configurations.

Also shown in FIG. 3A, the delivery vehicle 131 can also include a natural compound 133 absorbed, impregnated, embedded, or otherwise carried by the porous target product 111. In one example, the natural compound can include curcumin. In other examples, the natural compound can include Aloe Vera, Vitamin D, Vitamin C, or other suitable natural products. In certain embodiments, a loading profile of the natural product 133 can be generally uniform in the target product 111. In other embodiments, a loading profile of the natural product 133 can vary spatially within the target product 111. For example, as shown in FIG. 3A, a loading value (e.g., weight per volume) of the natural product 133 in the interior portion 111b may be higher than that of the exterior portion 111b. In other examples, the loading value of the natural product 133 can vary longitudinally, radially, or in other suitable fashions for achieving a desired release profile for the natural compound 133.

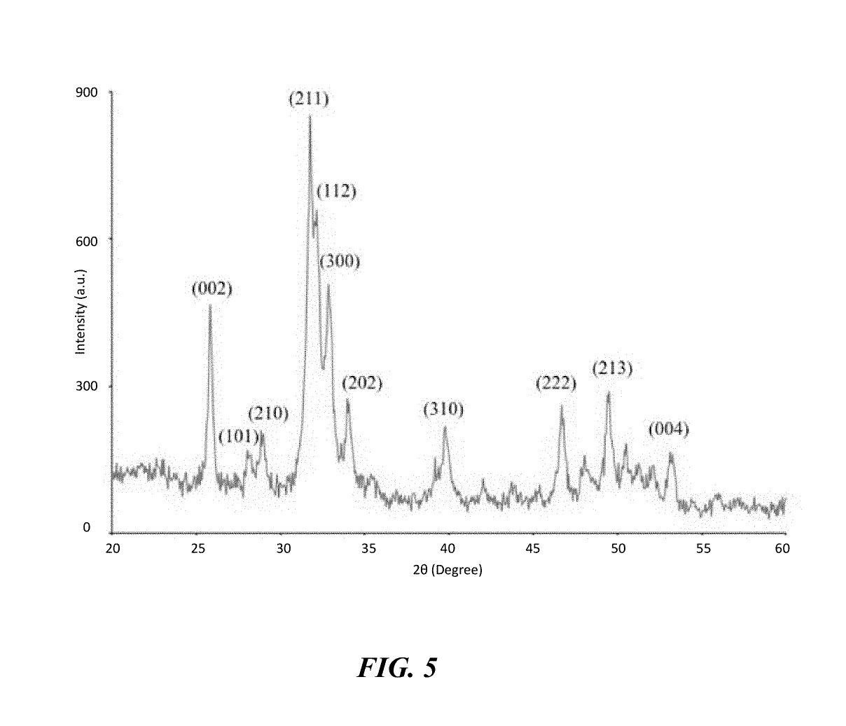

FIG. 3B shows another example delivery vehicle 131 in which the natural compound 133 is contained in a polymer matrix 135. In certain embodiments, the polymer matrix 135 can include one or more of PCL, PLGA, PEG, or other suitable polymeric materials. Without being bound by theory, it is believed that by adjusting a composition of the polymer matrix 135, bioavailability of the natural compound 133 may be increased than not using the polymer matrix 135. For example, it is believed that one or more of PCL and/or PLGA can be used to modify a hydrophilicity of curcumin such that curcumin can be readily absorbed into the circulatory system of a human or animal body. It is also believed that by using the polymer matrix 135, a desired release profile for the natural product 133 can be achieved, as described in more detail below with reference to FIGS. 5-11.

FIG. 3C shows another example delivery vehicle 131 in which the target product 111 has generally uniform porosity and has a barrier layer 137 applied to the surfaces of the target product 111. The barrier layer 137 can include PCL, PLGA, PEG, or other suitable compositions configured to affect a release rate of the natural compound 133 carried by the delivery vehicle 131. Though the barrier layer 137 is only shown on the exterior surface of the target product 111, in certain embodiments, the barrier layer 137 can also include portions that cover some or all of the pores in the target product 111.

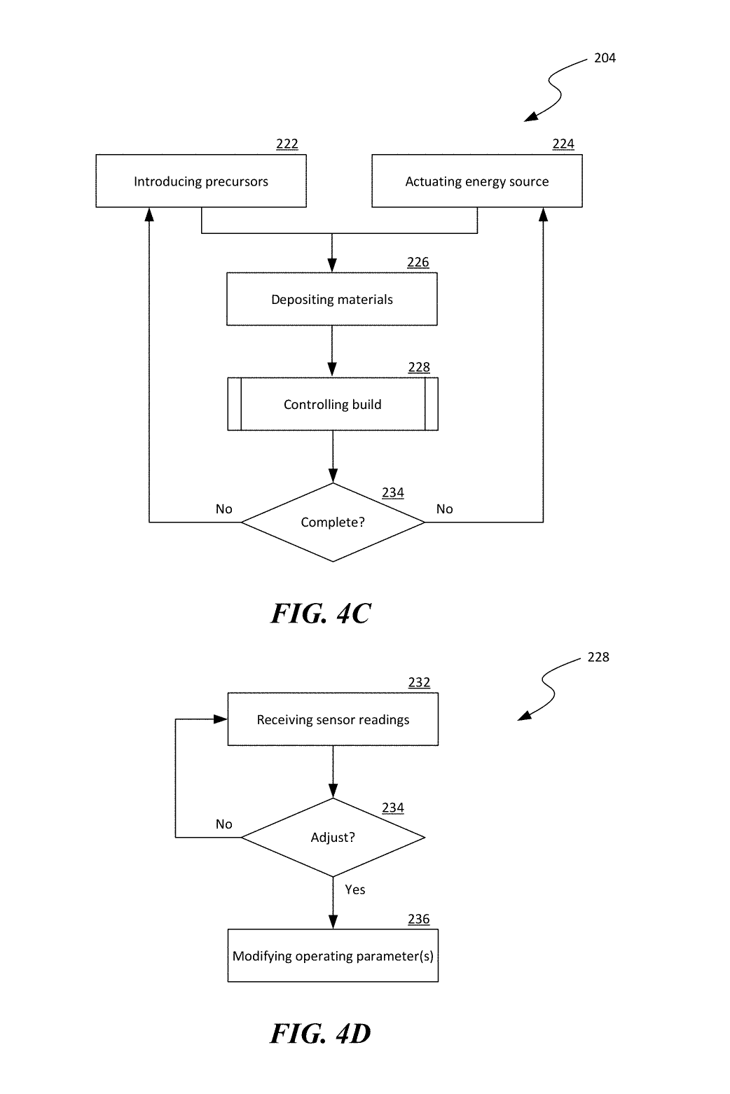

Though FIGS. 3A-3C illustrate particular examples of a delivery vehicle 131 configured according to the disclosed technology, in other embodiments, the delivery vehicle 131 can include other suitable arrangements for achieving desired release profiles. For example, the barrier layer 137 of FIG. 3C can also be applied to the target product 111 in either FIG. 3A or 3B. In another example, the delivery vehicle 131 of FIG. 3C can also carry the polymer matrix 135 of FIG. 3B. The various embodiments of the delivery vehicle 131 can be formed according to processes discussed below with reference to FIGS. 4A-4D.

In any of the foregoing embodiments, the delivery vehicle 131 can controllably release the carried natural compound 133 directly into the circulatory system of a human or animal body as the biodegradable ceramic material, ceramic-polymer composite, or ceramic-polymer-metal composite degrades when the delivery vehicle 131 is implanted in the human or animal body. Thus, a loading profile of the natural compound 133 in the delivery vehicle 131 can be adjusted to control an amount of the natural compound 133 releasable into the human or animal body at a given time point subsequent to implantation. For example, if the interior portion 111b in FIG. 3A contains higher concertation of the natural compound 133 than the exterior portion 111a, the delivery vehicle 131 can release or deliver higher dosage of the natural compound 133 after the exterior portion 111a has degraded after implantation. In other examples, the structure of the delivery vehicle 131 can also be used to control the amount of natural compound 133 releasable into the human or animal body even when the loading profile of the natural compound 133 is generally uniform in the delivery vehicle 131. For example, if the exterior portion 111a of the delivery vehicle 131 degrades slower than the interior portion 111b, an initial releasing rate of the natural compound 131 can be slower than a later releasing rate when the exterior portion 111a is completely or substantially completely degraded. In further examples, the loading profile of the natural compound 131, the structure and/or composition of the delivery vehicle 131, the application of the barrier layer 137 (or the lack thereof), and other suitable parameters may be adjusted in order to achieve a desired release profile of the natural compound 133 when the delivery vehicle 131 is implanted in the human or animal body.

FIG. 4A is a flowchart showing a method 200 for manufacturing an implantable delivery vehicle for sustained release of natural compounds in accordance with embodiments of the present technology. Even though the method 200 is described below with reference to the manufacturing system 100 of FIG. 1 and the software modules of FIG. 2, the method 200 may also be applied in other systems with additional or different hardware and/or software components.

As shown in FIG. 4A, the method 200 includes developing a build recipe at stage 202, for instance, utilizing the controller 120 of FIG. 1. In one embodiment, a build recipe can include a sequence of operations and operating parameters for each operation in the sequence. Example operating parameters can include feed rates of precursor materials from first and/or second feed lines 105a and 105b, power of the energy source 104, speed and direction of movement of the deposition platform 102, introduction of the precursor gas from the precursor gas source 113, and/or other suitable parameters. In other embodiments, a build recipe can include adjustment of operating parameters of sequential operations or other suitable information. Example operations of developing a build recipe are discussed in more detail below with reference to FIG. 4B.

The method 200 can also include performing a build via additive deposition based on the developed build recipe at stage 204. For example, in certain embodiments, one or more precursor materials in a determined proportion can be instructed into a deposition environment in which the precursor materials are melted and reacted with one another and/or with a substrate material to form a composite material. The formed composite material can then be allowed to solidify and deposited onto a substrate. The foregoing operations can then be repeated based on the developed build recipe until the product (FIG. 1) is completed. Example operations of performing a build based on the developed recipe are discussed in more detail below with reference to FIG. 4C.

The method 200 can also include applying a natural compound to the composite material at stage 206. In certain embodiments, applying the natural compound can include introducing a solution of the natural compound to the composite material via pipetting, soaking, baking, or other suitable techniques. In other embodiments, the natural compound can also be introduced to the composite material when carried in a polymer matrix of PCL, PLGA, PEG, or other suitable polymeric materials. In further embodiments, the natural compound can be introduced via gas- or liquid-phase infusion or other suitable techniques.

As shown in FIG. 4A, the method 200 can optionally include forming a barrier layer on the composite material loaded with the natural compound at stage 208. In certain embodiments, the barrier layer 208 can include one or more of PCL, PLGA, or PEG. In other embodiments, the barrier layer 208 can include other suitable materials. In further embodiments, the barrier layer 208 may be omitted.

FIG. 4B is a flowchart illustrating a process 202 of developing a build recipe in accordance with embodiments of the disclosed technology. As shown in FIG. 4B, the process 202 can include receiving a design file for the product at stage 212. In one embodiment, the design file can include a CAD file. In other embodiments, the design file can include any suitable types of file specifying a shape, composition, composition variation, dimension, or physical property of the product.

The process 202 can also include computing a recipe based on the received design file at stage 214. In one embodiment, computing the recipe can include constructing a sequence of operations to build the product in a layer-by-layer, section-by-section, or other suitable manners. Each operation sequence in the sequence can be associated with one or more operating parameters discussed above with reference to FIG. 4A.

FIG. 4C is a flowchart illustrating a process 202 of performing a build in accordance with embodiments of the disclosed technology. As shown in FIG. 4C, the process 202 can include introducing one or more precursor materials at stage 222 and actuating an energy source (e.g., a laser) at stage 224. Even though the operations at stages 222 and 224 are shown as concurrent in FIG. 4C, in other embodiments, these operations may be performed sequentially or in other suitable manners. The process 204 can also include deposition a composite material onto, for example, a substrate or unfinished product at stage 226.

The process 204 can further include controlling the build by varying one or more operating parameters based on the developed recipe at stage 228, as described in more detail below with reference to FIG. 4D. The process 204 can then include a decision stage to determine whether the build is completed. If the product is complete, the process 204 ends; otherwise, the process 204 reverts to introducing precursor materials at stage 222 and actuating laser scanning at stage 224.

FIG. 4D is a flowchart illustrating a process 228 of controlling a build in accordance with embodiments of the disclosed technology. As shown in FIG. 4D, the process 228 can include receiving sensor readings at stage 232. Example sensor readings can be from the feed rate sensors 119 of FIG. 1. The process 228 can then include a decision stage 234 to determine if adjustment is needed based on, for example, a comparison of the received sensor readings and the developed recipe. If adjustment is needed, the process 228 can include modifying the operating parameters at stage 236. For instance, at least one of the feed rates of the precursors can be modified such that a ratio of the precursor materials and a composition of a resulting composite material can be varied. In one example, the ratios of the precursor materials can be varied to result in a structure having composite materials with compositions transitioning from 100% metal to 100% ceramic. In another example, the ratios of the precursor materials can be varied to result in composite materials with compositions transitioning from 100% metal to 100% ceramic, and back to 100% metal. In further examples, the ratios of the precursor materials can be varied to result in composite materials with other suitable transitioning compositions.

Without being bound by theory, bones in a human or animal body can undergo dynamic remodeling, maturation, differentiation, and resorption that are controlled via interactions among osteocyte, osteoblast, and osteoclast cells. Although bone has self-healing abilities, large-scale bone defects cannot be healed completely by a human or animal body. Among different treatment options, bone tissue engineering focuses on methods to synthesize and/or regenerate bones to restore, maintain or improve osseous functions in vivo.

Human bones are porous with a gradient of interconnected porosity from the inside to the outside of a bone. Enhanced osseous tissue ingrowth into the interconnected porosity can improve mechanical interlocking between the neo-generated bone tissue and implanted scaffolds. Interconnected porosity can also facilitate exchange of blood and nutrients and removal of waste materials, which in turn can improve vascularization and accelerated bone healing. Bone tissue engineering scaffolds with interconnected porosity can also induce early stage osseo-integration by cell attachment and differentiation.

Calcium phosphate (CaP) ceramics have been used extensively for bone tissue engineering for no or low load bearing applications due to their indistinguishable compositions compared to natural bones and tunable bioresobability. Moreover, scaffold architectural features made out of CaP like volume fraction porosity, pore interconnectivity, pore size, shape can play a role in success of the scaffold in the living system.

Biodegradable ceramic scaffolds that exhibit controlled and sustained release of drugs or osteogenic factors for a desired period of time can facility effective medical treatment. Biodegradable polymers can be used to inhibit the burst release from CaP scaffolds. Due to its favorable mechanical properties and desired biodegradability, PCL can be used as a polymeric coating on scaffolds for bone tissue engineering and drug delivery applications. Controlled delivery of drug/protein/vitamin can affect the bone healing and remodeling process. Implantable ceramic scaffolds can utilize the advantages of local drug delivery system, bypassing the problems related to oral administration, for example, requirement for high dosage due to lack of vascularization in bone tissue.

The search for traditional natural compounds with chemo-therapeutic and chemo-preventive potential has motivated formulation of drug delivery systems such as implants. For example, curcumin, derived from the rhizomes of the turmeric plant, is believed to regulate the expression of genes involved in metastasis, cell proliferation, angiogenesis and osteoclastogenesis. These processes are associated with multiple cellular targets like nuclear factor and cyclooxygenase-2. It also induces apoptosis in oncogenic cells by suppressing variety of intracellular transcription factors and secondary messengers such as NF-kB, AP-1, c-Jun, and the JAK-STAT pathway. Curcumin exhibits anti-neoplastic properties, which inhibits or prohibits a variety of malignancies such as breast cancer, leukemia, kidney cancer, prostate cancer, colon cancer, melanoma and osteosarcoma. At a cellular level, curcumin can modulate important molecular targets involved in regulation of bone remodeling. It is believed that curcumin can cause apoptosis in osteoclasts, impedes osteoclastogenesis in RAW 264.7 cells, and hinders osteoclast formation by lowering the level of RANKL expression induced by IL-1α in BMSCs.

However, poor bioavailability and rapid rate of metabolism of curcumin restrains its application in practical studies. Detailed research on curcumin drug delivery systems has significantly improved the bioavailability issue. Still, the rapid drug metabolism can still be a difficulty. Moreover, in order to maintain an effective therapeutic concentration in a blood stream, a controlled release may be synthesized. Therefore a fabrication of curcumin loaded calcium phosphate implants can effectively be used as local drug delivery system for enhanced osteogenesis for various skeletal disorders or regeneration application.

Certain experiments were conducted to examine the in vivo in vitro controlled release of curcumin from calcium phosphate ceramics as an example delivery vehicle for providing natural compounds to a human or animal body. In vitro release experiments revealed controlled curcumin release from polymeric curcumin loaded ceramic scaffolds. On the other hand, uncontrolled release associated with burst release was observed from only curcumin incorporated samples. As discussed in more detail below, experiments have shown that curcumin loaded in 3D interconnected macro porous TCP scaffolds is effective for improving in vivo bone regeneration.

### Scaffold Fabrication for In Vitro Study.

HA powders (Monsanto, USA) were ball milled for 24 hours with ethanol and zirconia balls (ball:powder ratio was 3:1). After ball milling, the solvent was driven off at 60° C. Dried powders were then pressed into disk (12 mm diameter and 2 mm thickness) using a uniaxial press. 0.6 gm powders were measured for each disc and 4000 MPa pressure was applied uniaxially for not less than 2 minutes. Green scaffolds were then sintered at 1250° C. in a conventional muffle furnace for 2 hours.

### Scaffold Fabrication for In Vivo Study.

β-TCP powder was synthesized using the solid state synthesis method. In a sketch, 1 mole of calcium carbonate (CaCO3) and 2 moles of calcium phosphate dibasic (CaHPO4) were ball-milled for 2 hours and calcined at 1050° C. for 24 hours. The sintered powder was mixed further with 1.5 times by weight of 100% ethanol (200 proof, Decon Labs, PA) and 5 times by weight of zirconia balls for 6 hours. The powder was dried at 60° C. for 96 hours. Cylindrical scaffolds (3.2 mm diameter by 5 mm height) were fabricated using a 3D printer (ProMetal®, ExOne LLC, Irwin, Pa., USA) with 3D interconnected square shaped macro pores of 400 μm. After the fabrication of the green ceramic parts, the aqueous based binder was cured at 175° C. for 1.5 hours to mold a green ceramic scaffold. After the removal of the loosely adhered powder by successive rounds of dry ultrasonication and air blowing, the final scaffolds are made by sintering at 1250° C. in a conventional furnace for 2 hours.

### Drug-Polymer Coating for In Vitro Study

Two different polymer solutions were prepared. One with PCL and PEG in 65:35 ratio, and another with PLGA and PEG in 65:35 ratio. Both polymer solutions were dissolved in Dichloromethane (DCM). A drug solution was prepared by dissolving curcumin at a drug load of 0.1% (w/v) in ethanol. 20 μl of drug solution was added on top of both surfaces of the HA discs so that the total drug amount reaches 40 μg in each discs. To drive off the solvent, the HA discs loaded with curcumin were kept at room temperature overnight. Subsequently, 20 μl of the polymer solution was added to the discs and solvents were evaporated at room temperature.

### Drug-Polymer Coating Technique for In Vivo Study

The drug/polymer coating applied to the calcium phosphate (CaP) scaffolds comprised of poly-ε-caplrolactone (PCL, 14,000 M.W.) and polyethylene glycol (PEG, 3,000 M.W.) at a total of 5 wt %, and 65:35 molar percent with respect to each other, as well as curcumin at a concentration of 1 mg/mL in anhydrous ethanol. In order to coat the calcium phosphate scaffolds with poly-ε-caplrolactone/polyethylene glycol/curcumin (PCL+PEG+Cur), 50 μs of drug/polymer solution were pipetted onto each of the four sides of the scaffold until the desired drug loading was achieved.

### Pore Size, Coating Morphology, and Mechanical Properties

Field-emission scanning electron microscope (“FESEM”) (FEI Inc., Hillsboro, Oreg., USA) was utilized to analyze surface morphology of the scaffolds and measure the pore size of the fabricated scaffolds, following a gold sputter-coating (Technics Hummer V, CA, USA). Pore size after sintering was calculated by averaging the measurements for pure β-TCP scaffolds (n=3).

### In Vitro Curcumin Release

The release behavior of curcumin was studied in two different pH buffer solutions. A pH 7.4 phosphate buffer was used to emulate the physiological pH and pH 5.0 acetate buffer to emulate the post-surgical acidic microenvironment. pH measurements were done using a pH probe and within ±0.05. Three disc samples from each parameter were placed in 4 mL of pH 7.4 phosphate buffer and pH 5 acetate buffer in separate vials. The samples were kept at 37° C. under constant shaking of 150 rpm. The buffer solutions were collected at 1, 2, 4, 6, 9, 12, 24, 48, 72, 96, 120, 144, 240 and 360 hour time points. For each time point the solution of the vial was replaced by a freshly prepared 4 mL pH buffer solution. The concentrations of curcumin were analyzed by a UV-Vis spectrophotometer at 427 nm wavelength in a Biotek Synergy 2 SLFPTAD microplate reader (Biotek, Winooski, Vt., USA).

Change in Phase, Dissolution, and Surface Morphology after Release

Phase analyzes of the scaffolds were assessed by XRD using a Philips PW 3040/00 Xpert MPD system (Philips, Eindhoven, The Netherlands) with Cu Kα radiation and Ni filter. Scanning range of 20° to 60° at a step size of 0.1° and a count time of 1 s per step were applied. After the release study the scaffolds were air dried at ambient temperature for 72 hour and surface morphologies were observed under a field emission scanning electron microscope as described above. Fourier transform infrared (FTIR) spectra in the wave number range of 400-4000 cm-1 were analyzed to observe the functional groups present in the sample using an ATR-FTIR spectrophotometer (Nicolet 6700 FTIR, Madison, Wis., USA).

### Surgery and Implantation Procedure

Skeletally mature male Sprague-Dawley rats (weight 280-320gms) were used in this study with handling and housing as per Institutional Animal Care and Use Committee (“IACUC”). A combination of isoflurane and oxygen was used during the anesthesia. Following shaving and disinfection by 5% iodine, all animals underwent a bilateral surgery and an critical size intramedullary defect was created in the distal femur (3 mm diameter by 5 mm in length). The fabricated scaffolds were implanted in the defect by a press fit method. Un-doped TCP scaffolds were used as controls in the left femur while curcumin coated scaffolds were implanted in the right femur to analyze the effects of curcumin release on the in vivo bone formation. A total of 3 rats (6 samples) were used in this study.

### Histo-morphology

At necropsy, all samples were immediately fixed in 10% neutral-buffered formalin (NBF) and then processed for histologic assessment with series of ethanol and acetone. 2 samples from each treatment group at each time point were processed for bone histology and embedded in spurs resin after a series of ethanol and acetone dehydration. Ground thin sections around 200 □m were prepared using diamond saw and stained with Modified Masson Goldner's trichrome stain and observed under a light microscope. The stain results in a greenish color for the mineralized bone and reddish-orange for the osteoid formation.

The other 2 samples from the same treatment group were preserved in 14% ethylenediaminetetraacetic acid (EDTA) for 8 to 10 weeks, until the osseous tissue softens and cut into thin sections of 10 to 20 μm. Stained slides were used to evaluate biocompatibility, osteogenesis and angiogenesis.

### Histo-Morphometric Analysis

Image J software was used for newly generated bone area (desired bone area normalized over the area of the entire tissue section, %). Bone area was analyzed from 1 mm width by 1 mm height tissue sections (n=6). Three optical microscope images from each rat totaling six images from two rats were used for histomorphometric analysis.

### Results and Discussion

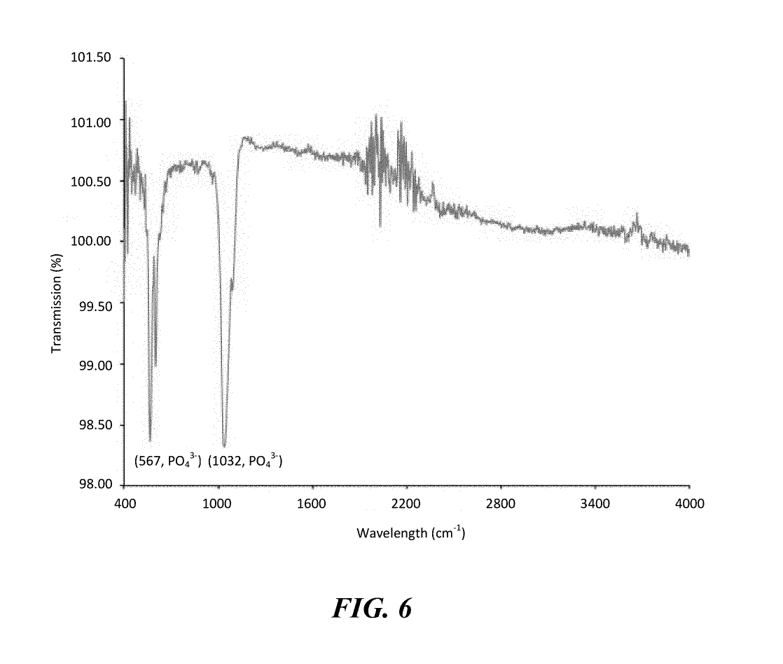

FIG. 5 shows XRD patterns of the HA powder used to prepare the discs. The sharp peaks refer to presence of crystalline HA (JCPDS no. 00-009-0432). Absence of other impurities in the XRD pattern indicates that the main phase of the substrate is hydroxyapatite crystal. FIG. 6 shows a FTIR spectrum that depicts the characteristic absorption peaks of the HA powder. The sharp peak at 632 cm-1 corresponds to the O—H deformation mode. Two bands appear at 567 and 1032 cm-1 were assigned to the bending vibrations of P043- in hydroxyapatite.

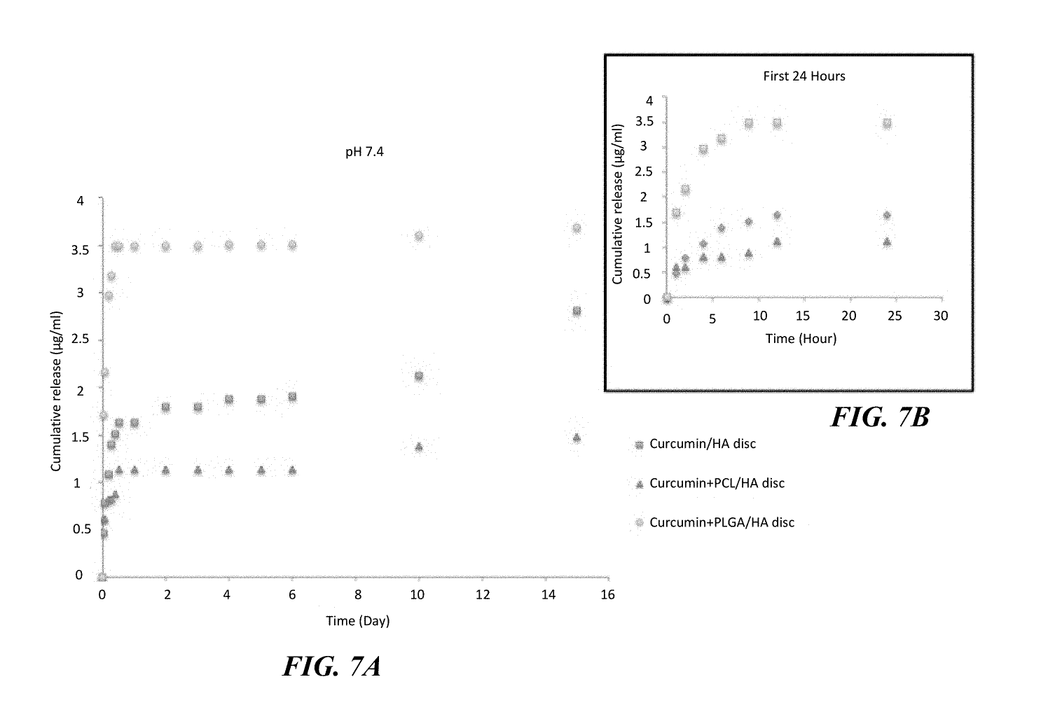

FIGS. 7A and 7B show curcumin release profiles from a) bare and b) PCL-coated (c) PLGA-coated HA discs at a phosphate buffer. In pH 7.4, a burst release of curcumin was observed for the PLGA coated HA discs within first 6 hours leading to a plateau. The degree of crystallization of PLGA decreased when crystalline PGA is co-polymerized with PLA. This appeared to lead to a higher rate of hydration and hydrolysis. 50:50 ratio of PLA/PGA resulted in the fastest degradation. The biodegradation of PLGA is believed to occur through the hydrolysis of the ester linkage. The drug release is still more controlled in case of polymer-coated samples than the uncoated ones. However, the total curcumin release was low (1.4, 2.8 and 3.6 μg release from PCL coated, uncoated and PLGA coated HA discs, respectively) in the phosphate buffer, which indicates that a majority of the curcumin had been degraded in neutral pH.

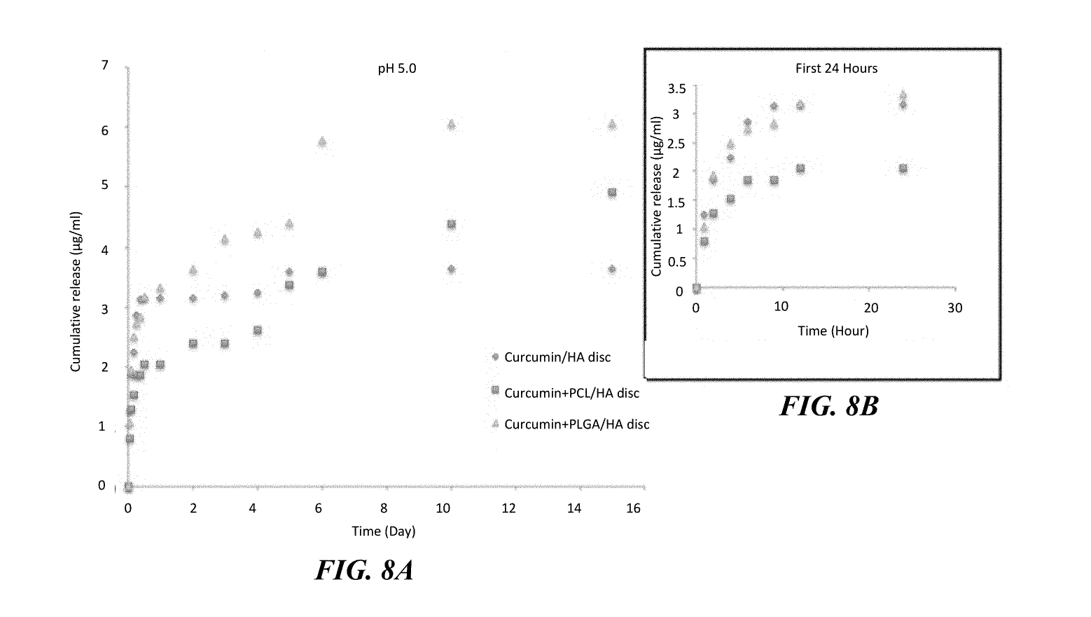

As shown in FIGS. 8A and 8B, PCL appeared to have inhibited the burst release of curcumin initially for pH 5.0 whereas both uncoated and PLGA coated HA discs showed burst release. Regardless of the polymer coating, cumulative curcumin release was higher (3.6, 4.3 and 6.0 μg release from uncoated, PCL coated, and PLGA coated HA discs, respectively) in pH 5.0 than pH 7.4. It is believed that pH of the release media influences the curcumin release kinetics from the polymer coated HA discs. Curcumin has higher stability in acidic pH conditions. On the other hand, proton is eliminated from the phenolic group in neutral-basic conditions.

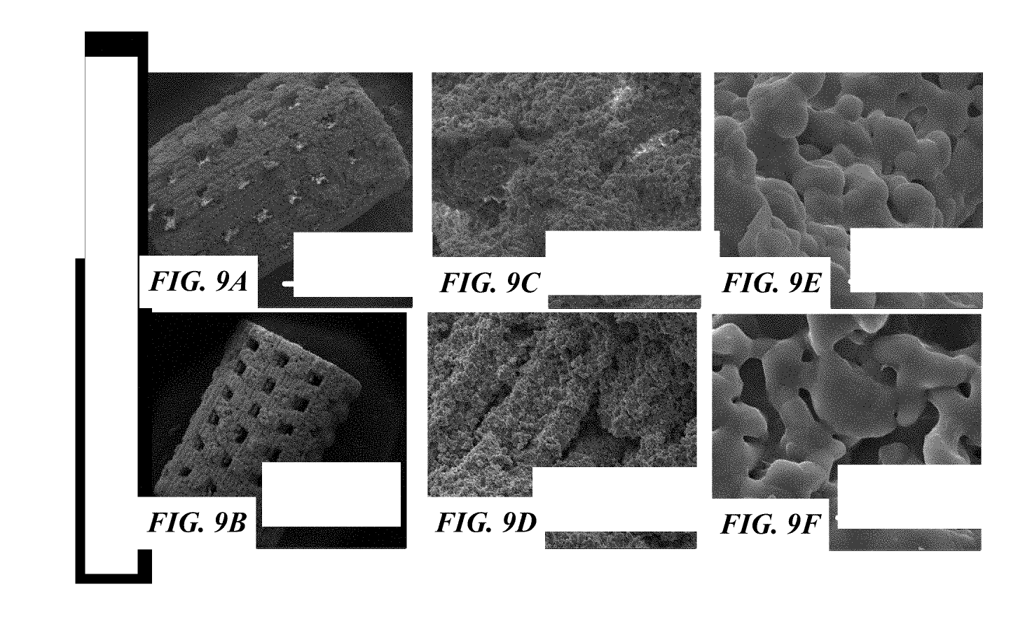

Three-dimensional printing technique enabled the direct fabrication of the complex scaffolds from calcium phosphate powders exhibiting the ability to print patient specific grafts for bone replacements. FIGS. 9A-9F show sintered pure and curcumin coated TCP scaffolds and surface morphology of the pure and curcumin coated TCP scaffolds. Intrinsic residual micro pores, around 20 μm, and designed macro pores were seen distributed uniformly on the walls of the scaffold in FIGS. 9A-9F. The intrinsic micro pores on the scaffold walls had been attributed to the absence of dense compaction process through the fabrication process. Sintered pore size has always been reported to be smaller than the designed pore size and is attributed to the densification during high temperature sintering process. Also, the total volume fraction porosity has been reported to be higher than the designed porosity. The sintered porosity in the pure TCP scaffolds was measured to be 350 μms.

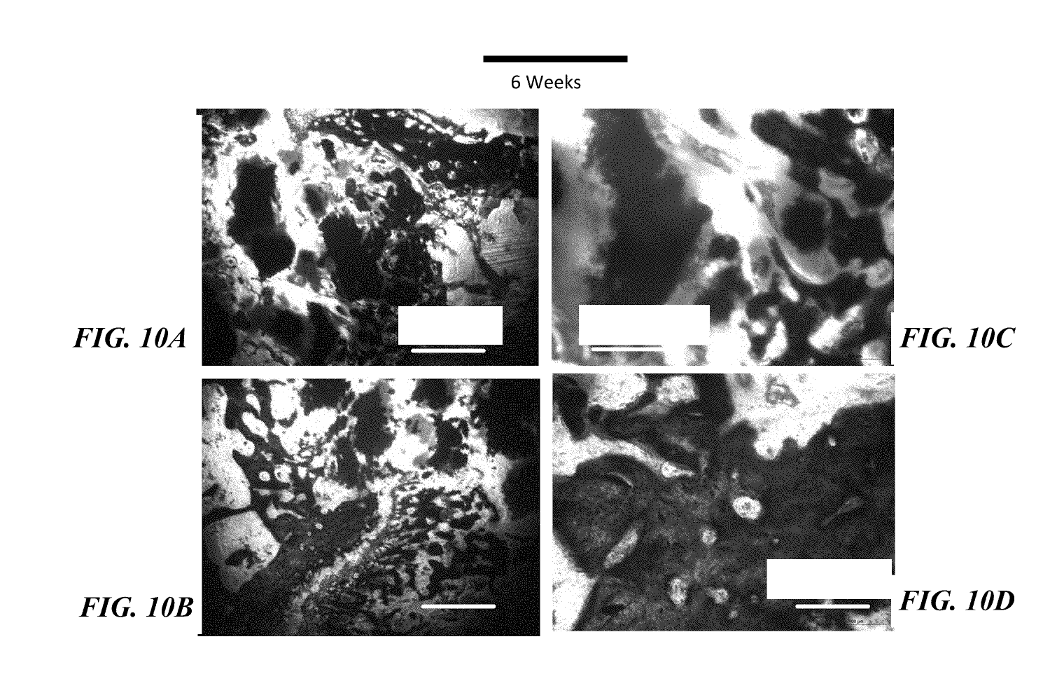

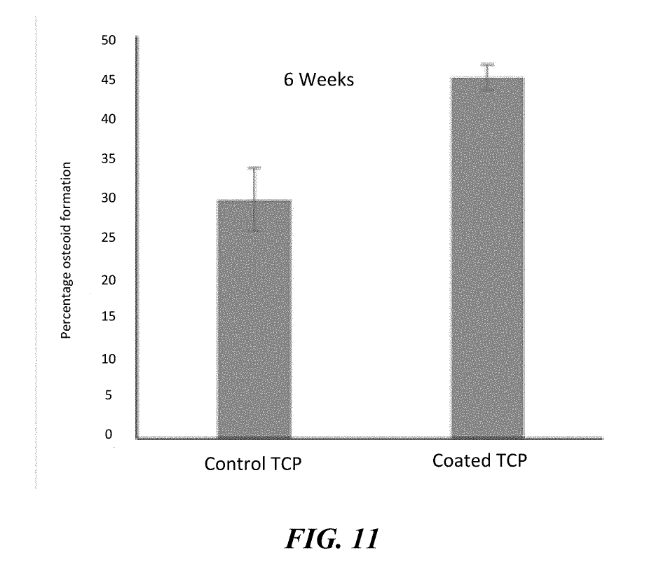

Influence of curcumin coating on biocompatibility of TCP and neo-bone formation was analyzed by histological evaluation at 6 weeks. FIGS. 10A-10D show the osteoid formation and mineralization of newly generated osseous tissue. Initiation of osteoid formation was revealed inside the designed macro-pores as well as the implant and host bone interface. A more complete mineralization of the newly formed bone and neo-bone integration was observed in curcumin TCP compared to bare TCP scaffolds as seen from FIGS. 10A-10D. The effect of curcumin coating in TCP on early wound healing with enhanced bone formation was prominently observed as shown in FIGS. 10A-10D. The multiscale porosity is believed to facilitate the infiltration of osteo-progenitor cells resulting in enhancement of new bone formation in both pure and coated TCP. Osteoid is the non-mineralized bone, which is formed during ECM protein secretion by bone forming osteoblast cells. The osteoid mineralization process was not complete in the control TCP whereas complete mineralization and osseo-integration was observed in curcumin coated TCP after 6 weeks. The increased bone formation induced by the presence of curcumin was furthermore substantiated by the significant difference in osteoid-formed area between pure and coated TCP as demonstrated by the histomorphometric analysis in FIG. 11.

Thus, as discussed above, experiments showed that a sustained and controlled release of curcumin was developed by using polymer coated hydroxyapatite substrates. Addition of PCL-PEG and PLGA-PEG co-polymer matrix enhanced bioavailability of curcumin as well as control release of the curcumin from the porous calcium phosphate ceramics. PCL coating inhibited the burst release of curcumin whereas PLGA coated and uncoated samples had burst release. 3D printing was utilized to fabricate pure and curcumin coated TCP scaffolds with designed porosity of 400 μms. The presence of curcumin in TCP scaffolds on early wound healing was clearly demonstrated by enhanced bone formation after 6 weeks compared to control TCP scaffolds. Complete mineralized bone formation increased from 29.6% to 44.9% in curcumin-coated TCP scaffolds compared to pure TCP scaffolds. Thus, interconnected macro porous TCP scaffolds loaded with curcumin can facilitate wound healing and tissue regeneration in bone tissue engineering.

From the foregoing, it will be appreciated that specific embodiments of the disclosure have been described herein for purposes of illustration, but that various modifications may be made without deviating from the disclosure. In addition, many of the elements of one embodiment may be combined with other embodiments in addition to or in lieu of the elements of the other embodiments. Accordingly, the technology is not limited except as by the appended claims.

We claim:

1. An implantable article of manufacture for enhancing bone healing in a human or animal body, comprising:
a three-dimensional scaffold implantable in the human or animal body, the three-dimensional scaffold containing a biodegradable ceramic material having multiple pores; and
curcumin embedded in the biodegradable ceramic material of the three-dimensional scaffold, the curcumin at least partially coating an exterior surface and the multiple pores of the biodegradable ceramic material of the three-dimensional scaffold, wherein the curcumin being directly releasable into a circulatory system of the human or animal body according to a target release profile as the biodegradable ceramic material degrades when the three-dimensional scaffold is implanted in the human or animal body.

2. The article of manufacture of claim 1, further comprising a biodegradable polymer coating on the three-dimension scaffold embedded with the curcumin, the biodegradable polymer coating including one or more of poly(ε-caprolactone), poly(lactic-co-glycolic acid), or poly-ethylene glycol, the biodegradable polymer coating having a composition corresponding to the target release profile of the curcumin when the three-dimensional scaffold is implanted in the human or animal body.

3. The article of manufacture of claim 1, further comprising a biodegradable polymer coating on the three-dimension scaffold embedded with the curcumin, the biodegradable polymer coating including poly(ε-caprolactone) for inhibiting burse release of the curcumin to the circulatory system of the human or animal body when the three-dimensional scaffold is implanted in the human or animal body.

4. The article of manufacture of claim 1, further comprising a biodegradable polymer coating on the three-dimension scaffold embedded with the curcumin, the biodegradable polymer coating including poly(lactic-co-glycolic acid) for enhancing burse release of the curcumin to the circulatory system of the human or animal body when the three-dimensional scaffold is implanted in the human or animal body.

5. The article of manufacture of claim 1, further comprising a biodegradable polymer coating on the three-dimension scaffold embedded with the curcumin, the biodegradable polymer coating including a mixture of poly(ε-caprolactone) and poly(lactic-co-glycolic acid) having a ratio corresponding to the target release profile of the curcumin when the three-dimensional scaffold is implanted in the human or animal body.

6. The article of manufacture of claim 1 wherein:
the biodegradable ceramic material having a first group of pores with a first size and a second group of pores of a second size different than the first size; and
the first and second groups are arranged spatially in the three-dimensional scaffold in accordance with the target release profile of the curcumin when the three-dimensional scaffold is implanted in the human or animal body.

7. The article of manufacture of claim 1 wherein the curcumin embedded in the biodegradable ceramic material is carried in a biodegradable polymer matrix having one or more of one or more of poly(ε-caprolactone), poly(lactic-co-glycolic acid), or poly-ethylene glycol.

8. The article of manufacture of claim 1 wherein:
the curcumin embedded in the biodegradable ceramic material is carried in a biodegradable polymer matrix having one or more of one or more of poly(ε-caprolactone), poly(lactic-co-glycolic acid), or poly-ethylene glycol; and
the article of manufacture further includes a biodegradable polymer coating on the three-dimension scaffold embedded with the curcumin, the polymer coating including one or more of poly(ε-caprolactone), poly(lactic-co-glycolic acid), or poly-ethylene glycol, the polymer coating having a composition corresponding to a target release profile of the curcumin when the three-dimensional scaffold is implanted in the human or animal body.

9. The article of manufacture of claim 1 wherein the biodegradable ceramic material includes (i) at least one of hydroxyapatite, β-tricalcium phosphate, calcium silicate, or calcium sulfate and (ii) an optional biodegradable polymer of one or more of poly(β-caprolactone), poly(lactic-co-glycolic acid), or poly-ethylene glycol.

10. A method of manufacturing an implantable device for directly deliver a natural compound to a human or animal body, comprising:
forming, via additive deposition, a three-dimensional scaffold implantable in the human or animal body, the three-dimensional scaffold being constructed from one or more of a porous biodegradable ceramic material, a ceramic-polymer composite of a biodegradable ceramic material and a polymer, or a ceramic-polymer-metal composite of a biodegradable ceramic material, a polymer, and a metal;
introducing a natural compound to be embedded in the formed three-dimensional scaffold, the natural compound at least partially coating an exterior surface of the three-dimensional scaffold; and
during introduction of the natural compound, varying a loading profile of the introduced natural compound in the three-dimensional scaffold according to a release profile such that the natural compound is directly and controllably releasable into a circulatory system of the human or animal body according to the release profile as the porous biodegradable ceramic material, the ceramic-polymer composite, or the ceramic-polymer-metal composite of the three-dimensional scaffold degrades when the implantable device is implanted in the human or animal body.

11. The method of claim 10 wherein forming the three-dimensional scaffold includes forming the three-dimensional scaffold containing the porous biodegradable ceramic material, the ceramic-polymer composite, or the ceramic-polymer-metal composite via 3D printing.

12. The method of claim 10, further comprising:
dissolving the natural compound in a biodegradable polymer matrix having one or more of one or more of poly(ε-caprolactone), poly(lactic-co-glycolic acid), or poly-ethylene glycol, a composition of the polymer matrix being selected according to the release profile; and
wherein introducing the natural compound includes introducing the natural compound carried in the biodegradable polymer matrix with the selected composition.

13. The method of claim 10, further comprising subsequent to introducing the natural compound, applying a biodegradable polymer coating having one or more of poly(ε-caprolactone), poly(lactic-co-glycolic acid), or poly-ethylene glycol to the three-dimensional scaffold embedded with the natural compound.

14. The method of claim 10, further comprising subsequent to introducing the natural compound, applying a biodegradable polymer coating having poly(ε-caprolactone) for inhibiting burse release of the natural compound to the circulatory system of the human or animal body when the implantable device is implanted in the human or animal body.

15. The method of claim 10, further comprising subsequent to introducing the natural compound, applying a biodegradable polymer coating having poly(lactic-co-glycolic acid) for enhancing burse release of the curcumin to the circulatory system of the human or animal body when the three-dimensional scaffold is implanted in the human or animal body.

16. The method of claim 10, further comprising:
selecting a ratio between poly(ε-caprolactone) and poly(lactic-co-glycolic acid) in a polymer matrix according to the release profile of the natural compound when the implantable device is implanted in the human or animal body; and
subsequent to introducing the natural compound, applying the biodegradable polymer coating having the selected ratio between the poly(ε-caprolactone) and the poly(lactic-co-glycolic acid)I to the porous ceramic material embedded with the natural compound.

17. A method of enhancing bone regeneration in a human or animal body, comprising:
implanting a three-dimensional scaffold in the human or animal body, the three-dimensional scaffold containing a porous ceramic material, a ceramic-polymer composite of a biodegradable ceramic material and a polymer, or a ceramic-polymer-metal composite of a biodegradable ceramic material, a polymer, and a metal and embedded with curcumin in the porous ceramic material, the ceramic-polymer composite, or the ceramic-polymer-metal composite, the curcumin at least partially coating an exterior surface of the three-dimensional scaffold; and
directly and controllably releasing the embedded curcumin into a circulatory system of the human or animal body according to a release profile as the porous ceramic material, the ceramic-polymer composite, or the ceramic-polymer-metal composite degrades subsequent to implanting the three-dimensional scaffold in the human or animal body, thereby achieving enhanced bone regeneration in the human or animal body.

18. The method of claim 17 wherein implanting the three-dimensional scaffold includes implanting the three-dimensional scaffold embedded with curcumin carried by a biodegradable polymer matrix having one or more of poly(ε-caprolactone), poly(lactic-co-glycolic acid), or poly-ethylene glycol.

19. The method of claim 17 wherein implanting the three-dimensional scaffold includes implanting the three-dimensional scaffold embedded with curcumin carried by a biodegradable polymer matrix having one or more of poly(ε-caprolactone), poly(lactic-co-glycolic acid), or poly-ethylene glycol, and wherein the three-dimensional scaffold also includes a barrier layer on the exterior surface of the three-dimensional scaffold, the barrier layer containing one or more of poly(ε-caprolactone) or poly(lactic-co-glycolic acid).

20. The method of claim 17 wherein implanting the three-dimensional scaffold includes implanting the three-dimensional scaffold embedded with the curcumin carried by a biodegradable polymer matrix having one or more of poly(ε-caprolactone), poly(lactic-co-glycolic acid), or poly-ethylene glycol and having a barrier layer on the exterior surface of the three-dimensional scaffold, the barrier layer containing one or more of poly(ε-caprolactone) or poly(lactic-co-glycolic acid).
